(19) United States
(12) Patent Application Publication
(10) Pub. No.: US 2003/0018071 A1
(43) Pub. Date: Jan. 23, 2003

(54) Method and compositions for treating fibrotic diseases

(76) Inventors: Stephen I. Rennard, Omaha, NE (US); Tadashi Kohyama, Omaha, NE (US)

Correspondence Address: SMITHKLINE BEECHAM CORPORATION, CORPORATE INTELLECTUAL PROPERTY-US, UW2220, P. O. BOX 1539, KING OF PRUSSIA, PA, 19406-0939, US

(21) Appl. No.: 10/203,583

(22) PCT Filed: Feb. 15, 2001
(86) PCT No.: PCT/US01/04797

(51) Int. Cl. A61K 31/277

(52) U.S. Cl. 514/521

(57) ABSTRACT

This invention relates to compositions and methods for preventing or treating fibrotic diseases by administering a phosphodiesterase 4-specific inhibitor as exemplified herein.

### AREA OF THE INVENTION

This invention relates compositions and methods for preventing or treating fibrotic diseases by administering a phosphodiesterase 4-specific inhibitor as exemplified herein.

### BACKGROUND OF THE INVENTION

Fibroblasts are the major source of extracellular connective tissue matrix, and the recruitment and activation of these cells is thought to play an important role in both wound healing and in the development of fibrosis. Agents which could block fibroblast accumulation could play a therapeutic role in modulating fibrosis. It has been found that certain PDE4 inhibitors play a therapeutic role in modulating fibrosis and fibroproliferative diseases.

### SUMMARY OF THE INVENTION

This invention relates to a method for preventing or treating a fibrotic disease in a mammal by administering to a patient in need thereof an effective amount of a PDE 4-specific inhibitor which has a certain therapeutic ratio as defined herein.

### DETAILED DESCRIPTION OF THE INVENTION

The therapy contemplated by this invention comprises administering a PDE4-specific inhibitor to prevent the onset of a fibrotic disease or to treat a fibrotic disease. These disorders can be endogenously occurring, e.g. in sclerodermia, caused by genetic abnormalities, e.g. in certain forms of neonatal liver fibrosis, due to environmental agents (infectious/occupational/toxic/traumatic), such as in liver cirrhosis or lung fibrosis or post-burn scaring, or the tissue response to medical interventions, such as restenosis of blood vessels after angioplasty, or lung fibrosis after cancer therapy, or scaring after eye surgery. Other fibrotic disorders include but are not limited to hepatic fibrosis and cirrhosis, renal fibrosis, myelofibrosis, and keloids; or fibrosis that may occur as a result of damage or injury to cardiac tissue or the cardiovascular system.

Pneumoconioses is a group of diseases characterized by a diffuse fibrotic reaction in the lungs induced by the inhalation of organic or inorganic particulates and chemical fumes and vapors. The pathogenesis of the fibrosis is through the release of fibrogenic chemical mediators. Examples are coal worker's pneumoconiosis, silicosis, asbestosis, and bagassoisis. Metallic fumes of tin, iron, and beryllium are fibrotic agents.

Fibroblasts are the major source of extracellular connective tissue matrix, and the recruitment and activation of these cells is thought to play an important role in both wound healing and in the development of fibrosis. Agents which could block fibroblast accumulation could play a therapeutic role in modulating fibrosis. It has been found that PDE4 inhibitors play a therapeutic role in modulating fibrosis. It has also been found that PDE4-specific inhibitors like the ones discribed herein, to the exclusion of PDE3 and PDE5 inhibitors, attenuate significantly the degradation of collagen, particularly the degradation attributed to TNF-α and human nutreophil elastase-stimulated degradation.

The PDE4-specific inhibitor useful in this invention may be any compound that is known to inhibit the PDE4 enzyme or which is discovered to act in as PDE4 inhibitor, and which are only PDE4 inhibitors, not compounds which inhibit other members of the PDE family as well as PDE4. Generally it is preferred to use a PDE4 antagonists which has an IC₅₀ ratio of about 0.1 or greater as regards the IC₅₀ for the PDE IV catalytic form which binds rolipram with a high affinity divided by the IC₅₀ for the form which binds rolipram with a low affinity.

PDE inhibitors like theophylline and pentoxyfyllin inhibit all or most all PDE isozymes indiscriminately in all tissues. These compounds exhibit side effects, apparently because they non-selectively inhibit all PDE isozyme classes in all tissues. The target disease may be effectively treated by such compounds, but unwanted secondary effects may be exhibited which, if they could be avoided or minimized, would increase the overall therapeutic effect of this approach to treating certain diseases. For example, clinical studies with the selective PDE 4 inhibitor rolipram, which was being developed as an antidepressant, indicate it has psychotropic activity and produces gastrointestinal effects, e.g., pyrosis, nausea and emesis.

For purposes of this disclosure, the cAMP catalytic site which binds R and S rolipram with a low affinity is denominated the “low affinity” binding site (LPDE 4) and the other form of this catalytic site which binds rolipram with a high affinity is denominated the “high affinity” binding site (HPDE 4). This term “HPDE4” should not be confused with the term “hPDE4” which is used to denote human PDE4.

Initial experiments were conducted to establish and validate a [³H]R-rolipram binding assay. Details of this work are given in Example 1 below.

To determine whether both the high affinity binding activity and the low affinity binding activity resided in the same gene product, yeast were transformed by known methods and the expression of recombinant PDE 4 was followed over a 6 hour fermentation period. Western blot analysis using an antibody directed against PDE 4 indicated that the amount of PDE 4 expressed increased with time, reaching a maximum after 3 hour of growth. In addition, greater than 90% of the immunoreactive product was in the high speed (100,000×g) supernatant of yeast lysates. [³H]R-Rolipram binding and PDE activity were monitored along with protein expression. PDE 4 activity was co-expressed with rolipram-binding activity, indicating that both functions exist on the same gene product. Similar to results with the Western plot analysis, greater than 85% of the rolipram-inhibitable PDE activity and [³H]-rolipram binding activity was found to be present in the yeast supernatant fraction.

Overall, most of the recombinant PDE 4 expressed in this system exists as LPDE 4 and only a small fraction as HPDE 4. Consequently, inhibition of recombinant PDE 4 catalytic activity primarily reflects the actions of compounds at LPDE 4. Inhibition of PDE 4 catalytic activity can thus be used as an index of the potency of compounds at LPDE 4. The potency of compounds at HPDE 4 can be assessed by examining their ability to compete for [³H]R-rolipram. To develop SARs for both the low affinity and high affinity rolipram binding sites, the potencies of selected compounds were determined in two assay systems. Results from experiments using standard compounds were tabulated. As expected, certain compounds were clearly more potent in competing with [³H]R-rolipram at the site for which rolipram demonstrated high affinity binding as compared with the other site, the one at which rolipram is a low affinity binder. SAR correlation between high affinity binding and low affinity binding was poor and it was concluded that the SAR for inhibition of high affinity [³H]R-rolipram binding was distinct from the SAR for binding to the low affinity rolipram binding site.

It is now known that there are at least two binding forms on human monocyte recombinant PDE 4 (hPDE 4) with which inhibitors interact. One explanation for these observations is that hPDE 4 exists in two distinct forms. One binds the likes of rolipram and denbufylline with a high affinity while the other binds these compounds with a low affinity. The preferred PDE4 inhibitors of use in this invention will be those compounds which have a salutary therapeutic ratio, i.e., compounds which preferentially inhibit cAMP catalytic activity where the enzyme is in the form that binds rolipram with a low affinity, thereby reducing the side effects which apparently are linked to inhibiting the form which binds rolipram with a high affinity. Another way to state this is that the preferred compounds will have an IC₅₀ ratio of about 0.1 or greater as regards the IC₅₀ for the PDE 4 catalytic form which binds rolipram with a high affinity divided by the IC₅₀ for the form which binds rolipram with a low affinity.

A further refinement of this standard is that of one wherein the PDE4 inhibitor has an IC₅₀ ratio of about 0.1 or greater; said ratio is the ratio of the IC₅₀ value for competing with the binding of 1 nM of [³H]R-rolipram to a form of PDE 4 which binds rolipram with a high affinity over the IC₅₀ value for inhibiting the PDE IV catalytic activity of a form which binds rolipram with a low affinity using 1 microM[³H]-cAMP as the substrate. A further review explanation with of this test can be found in co-pending U.S. Pat. No. 5,998,428 the text of which is incorporated herein by reference to the extent that text is necessary to the practice of this invention.

Most preferred are those PDE4 inhibitors which have an IC₅₀ ratio of greater than 0.5, and particularly those compounds having a ratio of greater than 1.0. A preferred compound is cis 4-cyano-4-(3-cyclopentyloxy-4-methoxyphenyl)cyclohexan-1-carboxylic acid (Ariflo®). In addition, the following PDE4 inhibitors may be useful in the practice of this invention: AWD-12-281 from Astra (Hofgen, N. et al. 15th EFMC Int Symp Med Chem (Sept 6-10, Edinburgh) 1998, Abst P.98); a 9-benzyladenine derivative nominated NCS-613 (INSERM); D-4418 from Chiroscience and Schering-Plough; a benzodiazepine PDE4 inhibitor identified as CI-1018 (PD-168787; Parke-Davis/Warner-Lambert); a benzodioxole derivative Kyowa Hakko disclosed in WO 9916766; V-11294A from Napp (Landells, L. J. et al. Eur Resp J [Annu Cong Eur Resp Soc (September 19-23, Geneva) 1998] 1998, 12(Suppl. 28): Abst P2393); roflumilast (CAS reference No 162401-32-3) and a pthalazinone (WO 9947505) from Byk-Gulden; or a compound identified as T-440 (Tanabe Seiyaku; Fuji, K. et al. J Pharmacol Exp Ther, 1998, 284(1): 162).

Appropriate dosage results in improvement of the clinical disease and, in particular, causes an arrest or a reduction in the mass of the fibrocellular scar tissue or a decrease of the products it secrets, such as the levels of procollagen peptides in serum. The anatomical location of the fibroproliferative scar tissue to be treated is the single most important determinant for the mode of administration and the pharmaceutical preparation of these compounds. Hepatic fibrosis and cirrhosis require an orally active compound rapidly extracted by the liver from the portal blood leaving the gut. Pulmonary fibrosis, on the other hand, is more directly addressed by an intravenously administered agent possibly in combination with inhalation of an aerosolized compound, although some PDE4-specific inhibitors can be administered orally for treatment of pulmonary fibrosis. Vascular restenosis or cutaneous scarring can be managed by local application regimen.

The present compounds and pharmaceutically acceptable salts which are active when given orally can be formulated as syrups, tablets, capsules, controlled-release preparation or lozenges. A syrup formulation will generally consist of a suspension or solution of the compound or salt in a liquid carrier for example, ethanol, peanut oil, olive oil, glycerin or water with a flavoring or coloring agent. Where the composition is in the form of a tablet, any pharmaceutical carrier routinely used for preparing solid formulations may be used. Examples of such carriers include magnesium stearate, terra alba, talc, gelatin, acacia, stearic acid, starch, lactose and sucrose. Where the composition is in the form of a capsule, any routine encapsulation is suitable, for example using the aforementioned carriers in a hard gelatin capsule shell. Where the composition is in the form of a soft gelatin shell capsule any pharmaceutical carrier routinely used for preparing dispersions or suspensions may be considered, for example aqueous gums, celluloses, silicates or oils, and are incorporated in a soft gelatin capsule shell.

Typical parenteral compositions consist of a solution or suspension of a compound or salt in a sterile aqueous or non-aqueous carrier optionally containing a parenterally acceptable oil, for example polyethylene glycol, polyvinylpyrrolidone, lecithin, arachis oil or sesame oil.

Typical compositions for inhalation are in the form of a solution, suspension or emulsion that may be administered as a dry powder or in the form of an aerosol using a conventional propellant such as fluorinated hydrocarbons such as trichlorofluoromethane.

Preferably the composition for the PDE4 inhibitors is a unit dosage form such as a tablet or capsule, or a controlled release preparation.

The active ingredient may be administered from 1 to 6 times a day, sufficient to exhibit the desired activity. Preferably, the active ingredient is administered about once or twice a day, more preferably twice a day.

As for the amount of drug administered, it is believed that for the PDE4 inhibitors will be administered in an amount of between 1 and 200 micrograms per day per adult human.

The following examples are set out to illustrate, not limit, the invention described herein.

### EXAMPLE 1

### Attenuation of Fibroblast Chemotaxis Using Ariflo

It has been found that the phosphodiesterase 4 inhibitor cis 4-cyano4-(3-cyclopentyloxy-4methoxyphenyl)cyclohexan-1-carboxylic acid (Ariflo®) can attenuate fibroblast chemotaxis toward the chemoattractant fibronectin. Since the activity of a PDE4 inhibitor should be dependent on endogenous cyclic AMP levels, a study was designed to determine if endogenous prostaglandin production rendered cells susceptible to the effects of Ariflo. To accomplish this, human fetal lung fibroblasts were cultured and, after achieving confluence, were incubated for 60 minutes with and without indomethacin (2×10⁻⁶ M) to inhibit cyclooxygenase. Fibroblasts were then trypsinized and placed in the upper portion of a Boyden blindwell chemotaxis chamber. Fibronectin, 20 μg/ml, was placed in the lower portion of the chamber as the chemoattractant. Indomethacin (2×10⁻⁶ M) was added to the upper side of the chemotaxis chamber. Indomethacin alone resulted in a slight but variable stimulation of chemotaxis (159±33% compared to control p<0.05). Ariflo added to control fibroblasts inhibited chemotaxis in a concentration dependent manner. In three separate experiments, the concentration of Ariflo required to inhibit chemotaxis by 50% was 4.9±2.5 μg/ml. In the presence of indomethacin, Ariflo inhibited chemotaxis only at the highest concentration tested 10 μg/ml reducing the response to 58.7±21.0% of control (p<005) compared to indomethacin. The study therefore demonstrates that the PDE4 inhibitor Ariflo is dependent, at least in part, on endogenous cyclooxygenase activity and, presumably, PGE production in order to exert its inhibitory effect on fibroblast chemotaxis. That Ariflo is active even in the presence of indomethacin suggests alternate mechanisms for stimulating cyclic AMP also play a role. Since cyclooxygenase activity and prostaglandin production can be modulated by a variety of mediators present in an inflammatory milieu, fibroblasts in such a setting may be particularly sensitive to the inhibitory effects of Ariflo. Such an effect has therapeutic value in fibrotic disorders.

### EXAMPLE2

### Phosphodiesterase and Rolipram Binding Assays

### Example 2A

Isolated human monocyte PDE 4 and hrPDE (human recombinant PDE4) was determined to exist primarily in the low affinity form. Hence, the activity of test compounds against the low affinity form of PDE 4 can be assessed using standard assays for PDE 4 catalytic activity employing 1 microM [³H]cAMP as a substrate (Torphy et al., J. of Biol Chem., Vol. 267, No. 3 pp1798-1804, 1992).

Rat brain high speed supernatants were used as a source of protein and both enantionmers of [³H]-rolipram were prepared to a specific activity of 25.6 Ci/mmol. Standard assay conditions were modified from the published procedure to be identical to the PDE assay conditions, except for the last of the cAMP: 50 mM Tris HCl (pH 7.5), 5 mM MgCl₂, and 1 nM of [³H]-rolipram (Torphy et al., J. of Biol. Chem., Vol. 267, No. 3 pp1798-1804, 1992). The assay was run for 1 hour at 30° C. The reaction was terminated and bound ligand was separated from free ligand using a Brandel cell harvester. Competition for the high affinity binding site was assessed under conditions that were identical to those used for measuring low affinity PDE activity, expect that [³H]-cAMP and 5′ AMP were not present.

### Example 2B

### Measurement of Phosphodiesterase Activity

PDE activity was assayed using a [³H]cAMP SPA or [³H]cGMP scintillation proximity analysis (SPA) enzyme assay as described by the supplier (Amersham Life Sciences). The reactions were conducted in 96-well plates at room temperature, in 0.1 ml of reaction buffer containing (final concentrations): 50 mM Tris-HCl, pH 7.5, 8.3 mM MgCl12, 1.7 mM EGTA, [³H]cAMP or [³H] cGMP (approximately 2000 dpm/pmol), enzyme and various concentrations of the inhibitors. The assay was allowed to proceed for 1 hr and was terminated by adding 50 μl of SPA yttrium silicate beads in the presence of zinc sulfate. The plates were shaken and allowed to stand at room temperature for 20 min. Radiolabeled product formation was assessed by scintillation spectrometry. Activities of PDE3 and PDE7 were assessed using 0.05 μM [³H]cAMP, whereas PDE4 was assessed using 1 μM [³H]cAMP as a substrate. Activity of PDE1B, PDE1C, PDE2 and PDE5 activities were assessed using 1 μM [³H]cGMP as a substrate.

[³H]R-rolipram Binding Assay

The [³H]R-rolipram binding assay was performed by modification of the method of Schneider and co-workers, see Nicholson, et at., Trends Pharmacol. Sci., Vol. 12, pp. 19-27 (1991) and McHale et al., Mol. Pharmacol., Vol. 39, 109-113 (1991). R-rolipram binds to the catalytic site of PDE4 see Torphy et al., Mol. Pharmacol., Vol. 39, pp. 376-384 (1991). Consequently, competition for [³H]R-rolipram binding provides an independent confirmation of the PDE4 inhibitor potencies of unlabeled competitors. The assay was performed at 30° C. for 1 hr in 0.5 l buffer containing (final concentrations): 50 mM Tris-HCl, pH 7.5, 5 mM MgCl2, 0.05% bovine serum albumin, 2 nM [³H]R-rolipram (5.7×104 dpm/pmol) and various concentrations of non-radiolabeled inhibitors. The reaction was stopped by the addition of 2.5 ml of ice-cold reaction buffer (without [³H]-R-rolipram) and rapid vacuum filtration (Brandel Cell Harvester) through Whatman GF/B filters that had been soaked in 0.3% polyethylenimine. The filters were washed with an additional 7.5-ml of cold buffer, dried, and counted via liquid scintillation spectrometry.

### EXAMPLE 3

### Preparation of a Controlled Release Tablet

A controlled-release formulation was prepared using the ingredients set out in Table

TABLE 1
Table Ingredients

| Ingredient | % w/w |
|---|---|
| Ariflo ® | 3.3 |
| Dibasic Calcium Phosphate | 88.5 |
| (anhydrous) | |
| Carbomer 934P | 3.3 |
| Carbomer 941P | 1.6 |
| Magnesium Stearate | 1.0 |
| Opadry White OY-S-9603 | 2.4 |
| Purified water | q.s. |

Blending and Compression Techniques:

Blending

Excipients and drug were placed in a blender and mixed. The magnesium stearate was then added and mixed for an additional 3 minutes. During the blending process, excipients and drug were mixed, passed through a screen and then mixed again.

Compression

Approximately 350 mg of each mix was compressed into tablets. A target tablet strength of 10 kp was used.

Opadry White was suspended in the purified water and that suspension was used to coat the tablets; water was removed during the coating process an ddid not form part of the final product.

### EXAMPLE 4

### Preparation of an Immediate Release Tablet

Immediate release tablets were prepared by standard means and contained the ingredients set out in Table 2.

TABLE 2
Immediate Release Tablets

| | Quantity | Quantity | Quantity |
|---|---|---|---|
| Ingredients | (mg/tablet) | (mg/tablet) | (mg/tablet) |
| Ariflo ® | 5.0 | 10.0 | 15.0 |
| Lactose Monohydrate | 113.0 | 108 | 103 |
| Microcrystalline Cellulose | 70.0 | 70.0 | 70.0 |
| Sodium Starch Glycolate | 10.0 | 10.0 | 10.0 |
| Magnesium Stearate | 2.0 | 2.0 | 2.0 |
| Opadry White OY-S-9603 | 5.0 | 5.0 | 5.0 |
| Total Tablet Weight (mg) | 205.0 | 205 | 205 |

### EXAMPLE 5

### Treatment of Pneumoconoisis

A patient diagnosed with silicosis is given a controlled-release tablet containing 30 mg of Ariflo® prepared as per Example 2 twice daily. Treatment is continued for a time deemed by the attending physician to be adequate for treating the release of fibrogenic chemical mediators which cause pathogenesis.

What is claimed is:

1. A method for treating a patient at risk for developing a fibrotic disease wherein the method comprises administering an effective amount of a PDE4 inhibitor alone or mixed with a pharmaceutically acceptable carrier.

2. A method for treating a patient with a fibrotic disease wherein the method comprises administering an effective amount of a PDE4 inhibitor alone or mixed with a pharmaceutically acceptable carrier.

3. The method of claim 1 or 2 wherein the fibrotic disease is pulmonary fibrosis.

4. The method of any one of claims 1-3 wherein the patient does not have asthma or chronic obstructive pulmonary disease.

5. The method of claim 1 or 2 wherein the fibrotic disease is hepatic fibrosis

6. The method of claim 5 wherein the patient does not have asthma or chronic obstructive pulmonary disease.

7. The method of claim 1 wherein the fibrotic disease is renal fibrosis.

8. The method of claim 7 wherein the patient does not have asthma or chronic obstructive pulmonary disease.

9. The method of any one of claims 1 to 8 wherein the PDE4 inhibitor is cis 4cyano-4-(3-cyclopentyloxy-4-methoxyphenyl)cyclohexan-1-carboxylic acid or roflumilast.
